(19) United States
(12) Patent Application Publication
(10) Pub. No.: US 2024/0268699 A1
(43) Pub. Date: Aug. 15, 2024

(54) AUTOMATED QUANTITATIVE JOINT AND TISSUE ANALYSIS AND DIAGNOSIS

(71) Applicant: MEDX SPA, Santiago (CL)

(72) Inventors: Boris Alejandro Panes Saavedra, Santiago (CL); Carlos Ignacio Andrade De Bonadona, Santiago (CL); Javier Andres Urzua Legarreta, Santiago (CL)

(21) Appl. No.: 18/565,917

(22) PCT Filed: Jul. 29, 2022
(86) PCT No.: PCT/IB2022/057087

§ 371 Date: Nov. 30, 2023

Related U.S. Application Data
(60) Provisional application No. 63/260,550, filed on Aug. 25, 2021.

(51) Int. Cl. A61B 5/055; A61B 5/00; A61B 5/107; G06T 7/00; G06T 7/11

(52) U.S. Cl. CPC A61B 5/055; A61B 5/1073; A61B 5/4528; A61B 5/4878; G06T 7/0012; G06T 7/11; G06T 2207/10088; G06T 2207/20084

(57) ABSTRACT

A system and method are disclosed for an automated quantitative joint and tissue analysis. In some embodiments, the quantitative joint information may be determined from MRI images that may be segmented together to form three-dimensional images of a selected joint. Regions of interest may autonomously be defined for the three-dimensional images. Quantitative joint and tissue information may be determined based on the defined regions of interest and the three-dimensional images. The three-dimensional images and joint and tissue information may be displayed and viewed.

### FIELD

The methods and apparatuses described herein relate generally to body joints, and more particularly to visualization and diagnosis of joint disorders.

### BACKGROUND

Osteoarthritis (OA) is a leading cause of permanent disability. More than three hundred million people are diagnosed with OA worldwide. OA is a multifactorial degenerative joint disease that typically affects people over 65 years of age. OA causes joint stiffness, pain and permanent movement impairment. This degenerative joint disease has proven to be highly influenced by the incidence of joint tissue focal lesions of young population. OA is a leading cause of reduced and/or inhibited physical activity in older people and the need for aids such as a wheelchair or a cane to move independently.

Despite improvements in imaging technology, joint tissue focal lesions and OA diagnostics has been limited to manual image interpretation by a trained clinician, which may be prone to human error. Furthermore, joint damage may reach an advanced state before the damage becomes visible to the human eye.

Thus, it would be beneficial to autonomously process and analyze medical images of a patient to diagnose and track joint damage.

### SUMMARY OF THE DISCLOSURE

Described herein are systems and methods for determining and diagnosing joint tissue lesions including bone and cartilage. In general, magnetic resonance image data of a patient may be received. In some cases, complementary patient data such as demographic information, patient physical characteristics (weight, height, blood pressure, and the like) may also be received. The system may autonomously analyze the magnetic resonance image data and the complementary patient data to determine and display a diagnosis for a body joint of the patient.

In one embodiment, a method of determining joint tissue degeneration may include receiving a magnetic resonance imaging (MRI) data for a selected joint, generating MRI segments based at least in part on the MRI data, generating three-dimensional models based at least in part on the MRI segments, autonomously determining one or more regions of interest (ROIs) based at least in part on the three-dimensional models, generating three-dimensional diagnostic images illustrating selected tissue degeneration areas based at least in part on the three-dimensional models and the one or more ROIs, and displaying the three-dimensional diagnostic images.

In some embodiments, the one or more ROIs may be based at least in part on topological gradients of the three-dimensional models. Further, the topological gradients may be identified based on computer aided analysis of the three-dimensional models. In some other embodiments, the one or more ROIs may include three-dimensional bone regions near the selected joint. The three-dimensional bone regions may include a femur, a tibia, or a combination thereof.

In some examples, the one or more ROIs may include three-dimensional cartilage regions near the selected joint. The three-dimensional cartilage regions may include a femoral cartilage region, a tibial cartilage region, a tibial cartilage loading region, or a combination thereof. In some examples, the three-dimension diagnostic images may include a three-dimensional thickness map of a joint space associated with the selected joint. Determining the three-dimensional thickness map may include estimating an edge of one or more cartilage regions within an MRI segment associated with the selected joint, determining a skeleton associated with the selected joint, determining a volume based on the estimated edge and skeleton, and determining the thickness associated with the joint based on the volume, summed over the MRI segment.

In some examples, the three-dimensional diagnostic images may include a bone edema and inflammation image. The bone edema and inflammation image may be based at least in part on determining a water concentration in one or more tissues associated with the selected joint. In some other examples, the three-dimensional diagnostic images may include a joint space width image. The tabulated data associated with the three-dimensional diagnostic images may include the mean value computed from a lowest five percent distribution of joint spaces. The three-dimensional diagnostic images may include a bone spur identification image.

In some examples, the method of determining joint tissue degeneration may include determining a water concentration of bones and cartilage associated with the select joint based at least in part on determining a uniformity of voxel intensity. Furthermore, determining the uniformity may include determining an entropy associated with one or more three-dimensional models. In some other examples, determining the uniformity may include determining an energy associated with voxels of one or more three-dimensional models. In still other examples, determining the uniformity may include determining a gray level co-occurrence matrix of joint entropy. In another example, determining the uniformity includes determining a gray level co-occurrence matrix of inverse difference. The method of determining joint tissue degeneration may include determining quantitative joint information based at least in part on the three-dimensional models and displaying the quantitative joint information.

In some examples, the method of determining joint tissue degeneration may include predicting joint-related conditions based at least in part on the three-dimensional diagnostic images and displaying an image showing, at least in part, the predicted joint related-conditions. Furthermore, the predicting may include determining a classification of the predicted joint-related conditions. Also, the classifications may include pain progression, joint space width progression, pain and joint space width progression, neither pain nor joint space width progression, or a combination thereof. In some other examples, the prediction may be based on a deep-learning model executed by a trained convolutional neural network.

A system for determining joint tissue degeneration is disclosed. The system may include one or more processors and a memory configured to store instructions that, when executed by the one or more processors, cause the system to receive magnetic resonance imaging (MRI) data for a selected joint, generate MRI segments based at least in part on the MRI data, generate three-dimensional models based at least in part on the MRI segments, autonomously determine one or more regions of interest (ROIs) based at least in part on the three-dimensional models, generate three-dimensional diagnostic images illustrating selected tissue degeneration areas based at least in part on the three-dimensional models and the one or more ROIs, and display the three-dimensional diagnostic images.

In some embodiments, the one or more ROIs may be based at least in part on topological gradients of the three-dimensional models. The topological gradients may be identified based on computer aided analysis of the three-dimensional models. In some examples, the one or more ROIs may include three-dimensional bone regions near the selected joint. The three-dimensional bone regions may include a femur, a tibia, or a combination thereof.

In some examples, the one or more ROIs may include three-dimensional cartilage regions near the selected joint. The three-dimensional cartilage regions may include a femoral cartilage region, a tibial cartilage region, a tibial cartilage loading region, or a combination thereof. In some other examples, the three-dimension diagnostic images may include a three-dimensional thickness map of a joint space associated with the selected joint. Additionally, execution of the instructions may cause the system to estimate an edge of one or more cartilage regions within an MRI segment associated with the selected joint, determine a skeleton associated with the selected joint, determine a volume based on the estimated edge and skeleton, and determine the thickness associated with the joint based on the volume, summed over the MRI segment.

In some embodiments, the one or more ROIs may be based at least in part on topological gradients of the three-dimensional models. The topological gradients may be identified based on computer aided analysis of the three-dimensional models. In some examples, the one or more ROIs may include three-dimensional bone regions near the selected joint. Additionally, the three-dimensional bone regions may include a femur, a tibia, or a combination thereof. In some other examples, the one or more ROIs may include three-dimensional cartilage regions near the selected joint. The three-dimensional cartilage regions may include a femoral cartilage region, a tibial cartilage region, a tibial cartilage loading region, or a combination thereof.

In some examples, the three-dimension diagnostic images may include a three-dimensional thickness map of a joint space associated with the selected joint. Furthermore, execution of the instructions may cause the system to estimate an edge of one or more cartilage regions within an MRI segment associated with the selected joint, determine a skeleton associated with the selected joint, determine a volume based on the estimated edge and skeleton, and determine the thickness associated with the joint based on the volume, summed over the MRI segment.

In some examples, the three-dimensional diagnostic images may include a bone edema and inflammation image. The bone edema and inflammation image may be based at least in part on a determination of a water concentration in one or more tissues associated with the selected joint.

In some examples, the three-dimensional diagnostic images may include a joint space width image. Furthermore, the execution of the instructions may cause the system to determine a mean value from a lowest five percent distribution of joint spaces.

In some examples, the three-dimensional diagnostic images may include a bone spur identification image. In some other examples, execution of the instructions may cause the system to determine a water concentration of bones and cartilage associated with the select joint based at least in part on a determination of uniformity of voxel intensity. Additionally, the instructions to determine the water concentration may include instructions to determine an entropy associated with one or more three-dimensional models. In some variations, the instructions to determine the water concentration may include instruction to determine an energy associated with voxels of one or more three-dimensional models. In some other variations, the instructions to determine the water concentration may include instructions to determine a gray level co-occurrence matrix of joint entropy. In still other variations, the instructions to determine the water concentration may include instructions to determine a gray level co-occurrence matrix of inverse difference. In some examples, the execution of the instructions may cause the system to determine quantitative joint information based at least in part on the three-dimensional models and display the quantitative joint information.

In some examples, execution of the instructions may cause the system to predict joint-related conditions based at least in part on the three-dimensional diagnostic images and display an image showing, at least in part, the predicted joint-related conditions. Furthermore, the instructions to predict may further include instructions to determine a classification of the predicted joint-related conditions. The classifications may include pain progression, joint space width progression, pain and joint space progression, neither pain nor joint space width progression, or a combination thereof. In some other examples, the instructions to predict may be based on a deep-learning model executed by a trained convolutional neural network.

A non-transitory computer-readable storage medium comprising instructions that, when executed by one or more processors of a system, may cause the system to perform operations comprising receiving magnetic resonance imaging (MRI) data for a selected joint, generating MRI segments based at least in part on the MRI data, generating three-dimensional models based at least in part on the MRI segments, autonomously determining one or more regions of interest (ROIs) based at least in part on the three-dimensional models, generating three-dimensional diagnostic images illustrating selected tissue degeneration areas based at least in part on the three-dimensional models and the one or more ROIs, and displaying the three-dimensional diagnostic images.

In some examples, the one or more ROIs may be based at least in part on topological gradients of the three-dimensional models. Additionally, the topological gradients may be identified based on computer aided analysis of the three-dimensional models.

In some examples, the one or more ROIs may include three-dimensional bone regions near the selected joint. Additionally, the three-dimensional bone regions may include a femur, a tibia, or a combination thereof.

In some examples, the one or more ROIs may include three-dimensional cartilage regions near the selected joint. Additionally, the three-dimensional cartilage regions may include a femoral cartilage region, a tibial cartilage region, a tibial cartilage loading region, or a combination thereof.

In some examples, the three-dimension diagnostic images may include a three-dimensional thickness map of a joint space associated with the selected joint. Additionally, execution of the instructions may cause the system to estimate an edge of one or more cartilage regions within an MRI segment associated with the selected joint, determine a skeleton associated with the selected joint, determine a volume based on the estimated edge and skeleton, and determine the thickness associated with the joint based on the volume, summed over the MRI segment.

In some variations, the three-dimensional diagnostic images include a bone edema and inflammation image. Additionally, the bone edema and inflammation image may be based at least in part on a determination of a water concentration in one or more tissues associated with the selected joint.

In some examples, the three-dimensional diagnostic images may include a joint space width image. Additionally, execution of the instructions may cause the system to determine a mean value from a lowest five percent distribution of joint spaces.

In some examples, the three-dimensional diagnostic images may include a bone spur identification image. In some other examples, execution of the instructions may cause the system to determine a water concentration of bones and cartilage associated with the selected joint based at least in part on a determination of a uniformity of voxel intensity. Additionally, the determination of the uniformity may include a determination of an entropy associated with one or more three-dimensional models. In some variations, the determination of the uniformity may include a determination of energy associated with voxels of one or more three-dimensional models. In some other variations, the determination of the uniformity may include a determination of a gray level co-occurrence matrix of joint entropy. In still other variations, the determination of the uniformity may include a determination of a gray level co-occurrence matrix of inverse difference.

In some examples, execution of the instructions may cause the system to determine quantitative joint information based at least in part on the three-dimensional models and display the quantitative joint information.

In some examples, execution of the instructions may cause the system to predict joint-related conditions based at least in part on the three-dimensional diagnostic images and display an image showing, at least in part, the predicted joint-related conditions. Furthermore, the instructions to predict may further include instructions to determine a classification of the predicted joint-related condition. The classifications may include pain progression, joint space width progression, pain and joint space width progression, neither pain nor joint space width progression, or a combination thereof. In some other examples, the instructions to predict may be based on a deep-learning model executed by a trained convolutional neural network.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 illustrates an example system to autonomously analyze magnetic resonance imaging data and diagnose joint damage due to osteoarthritis.

FIG. 2 graphically depicts processing steps to generate joint diagnostic information from the MRI images of FIG. 1.

FIG. 3 shows segmented 3D images that may be used by the compute node of FIG. 1 to determine regions of interest associated with a femoral bone.

FIG. 4 shows an image illustrating the regions of interest of the femur based on regions of interest determined by the compute node as described above with respect to FIG. 3.

FIG. 5 shows an image illustrating the regions of interest associated with a femoral cartilage.

FIG. 6 shows segmented 3D images that may be used by the compute node of FIG. 1 to determine regions of interest associated with a tibial bone.

FIG. 7 shows an image illustrating the regions of interest associated with the tibia based on the ROIs described above with respect to FIG. 6.

FIG. 8 shows an image that may be used by the compute node of FIG. 1 to determine regions of interest associated with tibial cartilage.

FIG. 9 shows an image illustrating the regions of interest associated with the tibial cartilage as described above with respect to FIG. 8.

FIG. 10 shows an image illustrating the regions of interest associated with loading of the femoral cartilage.

FIG. 11 shows a series of images that may be used by the compute node of FIG. 1 to determine regions of interest associated with loading of the tibial cartilage.

FIG. 12 shows an image showing the regions of interest associated with a loaded tibial cartilage.

FIG. 13 shows an image showing a three-dimensional joint space width image based on the joint space determined from voxel data.

FIG. 14 shows an example femoral cartilage thickness map based on the extracted cartilage boundary and the skeletal representation, as described herein.

FIG. 15 shows an image with two arbitrary surfaces.

FIG. 16 shows an image depicting a surface distance map (e.g., a thickness map) associated with the two surfaces shown in FIG. 15.

FIG. 17 shows an image depicting bone edema/inflammation.

FIG. 18 shows an example image showing bone spurs.

FIG. 19 shows an example Entropy image where lighter areas indicate higher levels of randomness.

FIG. 20 shows an example energy image inside a femoral bone.

FIG. 21 shows an image depicting joint entropy within a femoral bone.

FIG. 22 shows an image 2200 depicting a gray level co-occurrence matrix inverse difference of a femoral bone.

FIG. 23 shows a diagnostic three-dimensional image depicting detected edema.

FIG. 24 is a flowchart depicting an example of one method for determining quantitative joint tissue degeneration measurements, in accordance with some embodiments.

FIG. 25 shows a block diagram of a compute node that may be an embodiment of the compute node of FIG. 1.

### DETAILED DESCRIPTION

Magnetic resonance image (MRI) data has become widely available, however diagnosing some joint diseases has proven to be difficult due to the nature of the MRI data. In some cases, the MRI images may include information and biomarkers that may assist in diagnosing many joint ailments, however the information and biomarkers may be difficult to extract with a visual inspection of the MRI data. A system and apparatus described herein can receive MRI data, process the MRI data to form three-dimensional models, define unique regions of interest based on the three-dimensional models and determine quantitative joint and tissue information based on the regions of interest and the three-dimensional models.

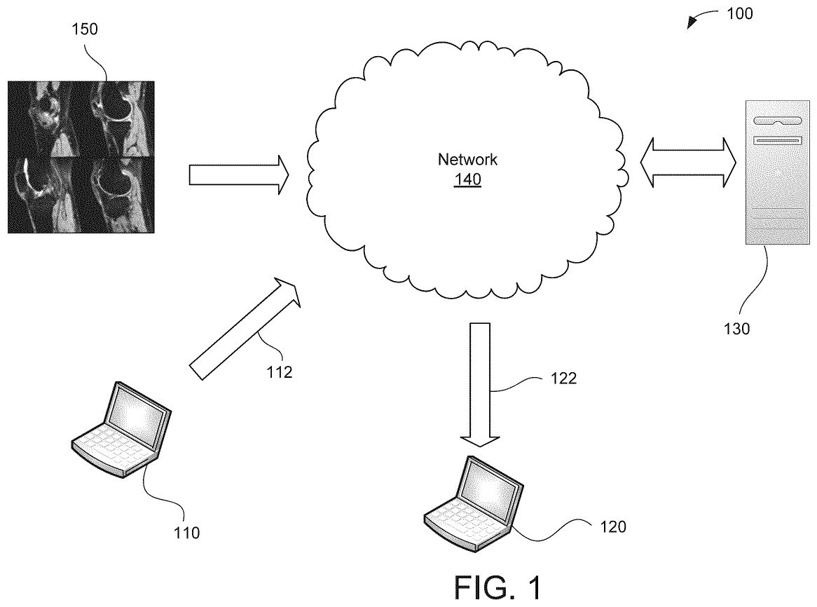

FIG. 1 illustrates an example system 100 to autonomously analyze magnetic resonance imaging (MRI) data and diagnose joint damage due to osteoarthritis. Throughout this application, reference will be made to the knee joint to explain concepts with concrete examples. Those having skill in the art will recognize that the methods and apparatus described herein is not limited to the knee, but instead may be applied to any feasible joint.

The system 100 may include an input terminal 110, an output terminal 120, a compute node 130, and a network 140. Although depicted as laptop computers, the input and output terminals 110 and 120 may be any feasible terminal and/or device such as a personal computer, mobile phone, personal digital assistant (PDA), other handheld devices, netbooks, notebook computers, tablet computers, display devices (for example, TVs, computer monitors, among others), among other possibilities. Similarly, the compute node 130 may be any feasible computing device such as a server, virtual server, blade server, stand-alone computer, a computer provisioned to run a dedicated, embedded, or virtual program that include one or more non-transitory instructions, or the like. For example, the compute node 130 may be a combination of two or more computers or processors. In some examples, the input terminal 110 and the output terminal 120 may be bidirectional terminals. That is, the input terminal 110 and the output terminal 120 may both transmit and receive data to and from the network 140. In some other examples, the input terminal 110 and the output terminal 120 can be the same terminal.

MRI images 150 may be sent through the network 140 to the compute node 130. The MRI images 150 may be captured by any feasible MRI device and may include images of a selected body joint to undergo analysis and/or diagnosis. In some examples, the MRI images 150 may be in a Digital Imaging and Communications in Medicine (DICOM) format. The network 140 may be any technically feasible network capable of transmitting and receiving data, including image data, numeric data, text data, database information, and the like. In some cases, the network 140 may include wireless (e.g., networks conforming to any of the IEEE 802.11 family of standards, cellular networks conforming to any of the LTE standards promulgated by the 3rd Generation Partnership Project (3GPP) working group, WiMAX networks, Bluetooth networks, or the like) or wired networks (e.g., wired networks conforming to any of the Ethernet standards), the Internet, or a combination thereof.

Additionally, a clinician may enter and transmit complementary patient data 112 at the input terminal 110 to the compute node 130 through the network 140. The complementary patient data 112 may include patient demographic information (e.g., gender, age, ethnicity), weight and height (or alternatively, a body mass index (BMI)), cohort, pain indication tests, semi-quantitative MRI based scores (e.g., whole-organ magnetic resonance imaging score (WORMS), Western Ontario and McMaster Universities (WOMAC) Osteoarthritis index, arthroscopic based evaluations (ICRS)), serum, urine-based analysis, or the like.

The compute node 130 may process and analyze the MRI images 150 and the complementary patient data 112 and determine an osteoarthritis diagnosis for the patient. The compute node 130 may also generate one or more images illustrating and/or highlighting joint damage that may not be discernible from the raw MRI images 150. The compute node 130 may transmit diagnosis information 122 (including any related generated images) to a clinician or patient through the network 140 to the output terminal 120. In some examples, the compute node 130 may generate prognostic information to predict the progression of any diagnosed joint damage. In some other examples, the compute node 130 may include a display (not shown) that may be used to display the generated images and/or the prognostic information.

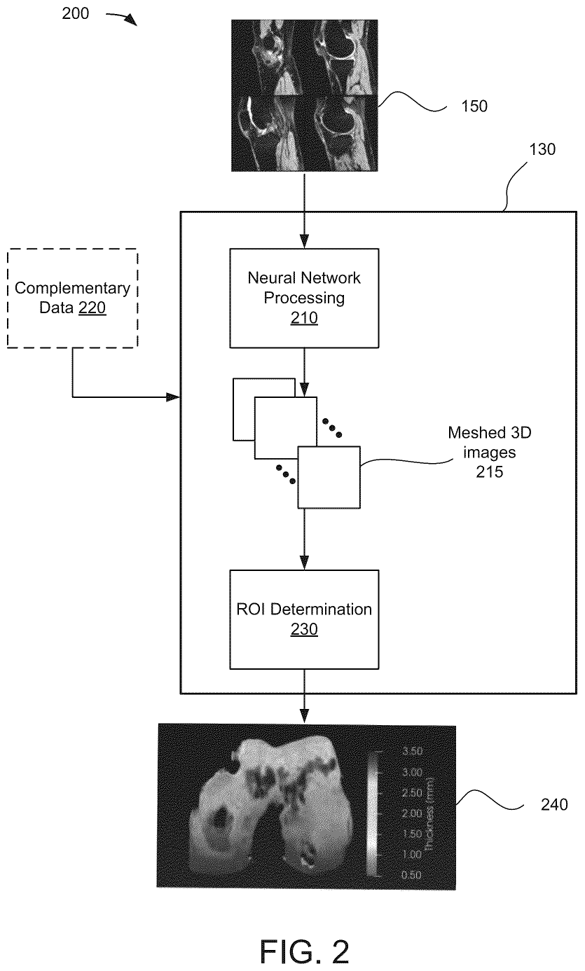

FIG. 2 graphically depicts processing steps 200 to generate joint diagnostic information from the MRI images 150 of FIG. 1. The MRI images 150 of a selected joint may be received by the compute node 130. Although shown and described herein as a knee joint, any feasible body joint may be selected and diagnosed.

The MRI images 150 may be processed with a neural network processing procedure 210. The neural network processing procedure 210 may be used to discriminate between different joint tissues and determine boundaries of each of the joint tissues. In some examples, the neural network processing procedure 210 may include a deep learning model based on a U-shaped convolutional neural network. The neural network may take a two-dimensional (2D) MRI image as an input with dimensions H×W×1 (where H is the height in pixels and W is the width in pixels) and output a segmented image with dimensions H×W×7. The seven (“7”) may correspond to the number of independent probability maps that are output to distinguish and/or define tissues and bones associated with the selected joint.

Returning to the example of the knee, the neural network processing procedure 210 may generate seven images: a femoral bone image, a femoral cartilage image, a tibial bone image, a tibial cartilage image, a patellar bone image, a patellar cartilage image, and a background image. Collectively, these images may be referred to as segmented images.

Next, the segmented images may be further processed to remove image artifacts and/or errors. Artifacts may include disconnected or floating portions of at least one of the segmented images. Such artifacts may be easily detected and removed. In some examples, artifact removal may be achieved with morphological operations that process the segmented images using a kernel (sometimes referred to as a filter kernel).

In addition, an upsampling algorithm may be used to provide shape refinement in 3D space that may improve both the anatomic representation of the joint in the space of segmented images and also allow a more precise quantification of geometric quantities such as volume, surface, thickness, etc. This process is especially useful and necessary when the input MRI sequences contain anisotropic voxels, which is the typical case in health centers nowadays. It is worth noticing that volumetric MRI sequences, or high-resolution sequences, contain high resolution quasi-isotropic voxels in three-dimensional space, while non-volumetric MRI sequences, or low-resolution sequences, only have quasi-isotropic and high-resolution pixels in one particular plane (sagittal, coronal or axial) and few samples (layers) in the orthogonal direction (low-resolution in the orthogonal direction), therefore containing highly anisotropic voxels. Indeed, in general it is expected that a typical MRI exam instead of producing a unique (expensive and time consuming) volumetric sequence it may produce several (cheap and fast) low-resolution MRI sequences, that consider complementary perspectives of the analyzed joint. For instance, a typical MRI exam may include sagittal, coronal and axial views of the joint. The upsampling algorithm proposed in this document uses as input the independent segmented images of these complementary views, which can be combined using a multi-planar combination model in order to produce a high resolution and unique volumetric representation of the joint tissues. This is achieved by following two sequential steps: first, a series of deterministic operations transform the independent segmentations that come from different planes into a common reference frame. These operations include a voxel isotropication process, which consists in a regular upscaling of the input images in order to generate enhanced representations with isotropic voxels, plus an image alignment process including affine image registration techniques that ultimately allows the anatomical superposition of different image plane views. Second, the multi-planar combination model comprising the application of a U-shaped fully convolutional neural network to the set of previously processed segmented planes in order to produce a unique high-resolution and quasi-isotropic volumetric representation. As a result, this multi-planar combination model may produce segmented three-dimensional (3D) images 215. Returning to the example of the knee, the segmented 3D images 215 may include a femoral bone, femoral cartilage, tibial bone, tibial cartilage, patellar bone, patellar cartilage and menisci.

Next, statistical shape modeling may be used to automatically select the side of the input knee sequence, when this information does not appear in the DICOM metadata, which is a highly relevant process to properly identify the regions of interest to be analyzed in the next step of the pipeline. Starting from a representative series of known high-quality and manually segmented images, knees with known side, two unique representations, one for each side (left and right), are obtained by considering the statistical average of these knees in the space of triangulated shapes. These baseline knees are adjusted to the input case and a loss function is computed. The proper side of the knee is determined by the case that minimize the loss function. It has been verified that this approach reaches 100% efficiency to automatically select the side of the input sequence.

Next, the compute node 130 may determine regions of interest (ROIs) 230. The ROIs may be determined with respect to the segmented 3D images 215. The ROIs may be autonomously determined without user guidance or intervention. The ROIs may be used in further processing steps to determine joint and/or tissue characteristics. For example, the compute node 130 may identify ROIs that include data and features that may be associated with particular segmented 3D images 215. In other words, the compute node 130 may provide a computer aided analysis of the segmented 3D images 215 to identify and determine ROIs. Since the ROIs are autonomously determined by the compute node 130, clinician subjectivity and error related to determining ROIs may advantageously be eliminated. Using the determined ROIs, the compute node 130 may generate (render) diagnostic 3D images 240 as well as determine quantitative measurements (e.g., diagnostic volumetric data) associated with the selected joint. The diagnostic 3D images 240 and quantitative measurements may then be displayed on an output terminal (such as output terminal 120). Note that the MRI images 150 and the diagnostic 3D image 240 depicted in FIG. 2 are merely to illustrate possible MRI images 150 and diagnostic 3D images 240 and are not meant to be limiting.

In some embodiments, complementary patient data 220 may optionally (shown by dashed lines) be transmitted to the compute node 130. The complementary patient data 220 may be used to aid patient diagnosis and/or prognosis.

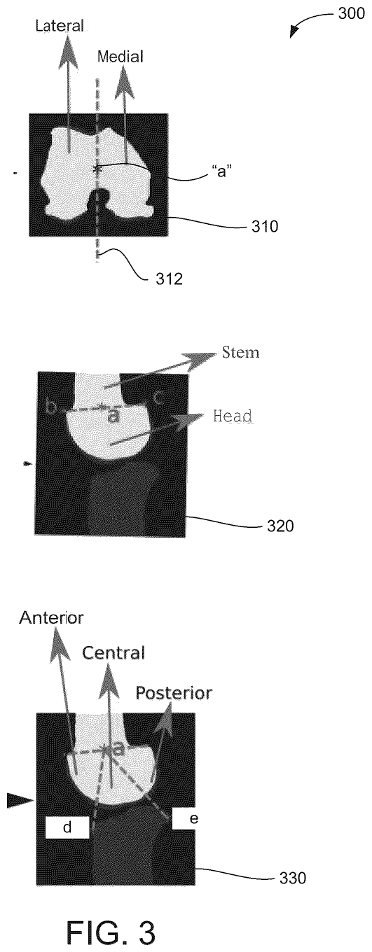

As described above, each of the diagnostic 3D images 240 may include two or more ROIs. For example, the compute node 130 may identify the data and features that may be associated with any of the segmented 3D images 215. The ROIs may assist in the determination of data, including quantitative measurements, that may be useful for diagnosing a current body joint condition and also for predicting a future body joint condition. Example procedures for determining ROIs for the diagnostic 3D images are discussed below with respect FIGS. 3 to 12. The procedures herein are described with respect to the knee joint in particular. Other body joints may have similar procedures.

FIG. 3 shows segmented 3D images 300 that may be used by the compute node 130 of FIG. 1 to determine ROIs associated with a femoral bone. Image 310 shows an axial view of a femur. The compute node 130 may determine a centroid “a” of the femur. The compute node 130 may project a vertical line 312 (e.g., a line in the antero-posterior direction) to divide the femur into lateral and medial portions. The lateral portion is shown on the left and the medial is shown on the right.

Next, stem and head portions may be determined. For example, the compute node 130, through computer aided analysis of segmented 3D images, may identify points “b” and “c” on the femur by identifying where a largest topological gradient (with respect to the outer surface of the femur) occurs. Image 320 shows a line that the compute node 130 may project from “b” to “c” passing through the centroid “a”. The stem portion may extend in a superior direction and the head may extend in an inferior direction with respect to the line from “b” to “c”.

Next, anterior, central, and posterior portions may be determined. Image 330 shows points “d” and “e” are identified on the tibia. These points are the most anterior and posterior (with respect to the outer surface) of the tibia. The compute node 130 may project a surface from point “d” to centroid “a” and point “e” to centroid “a”.

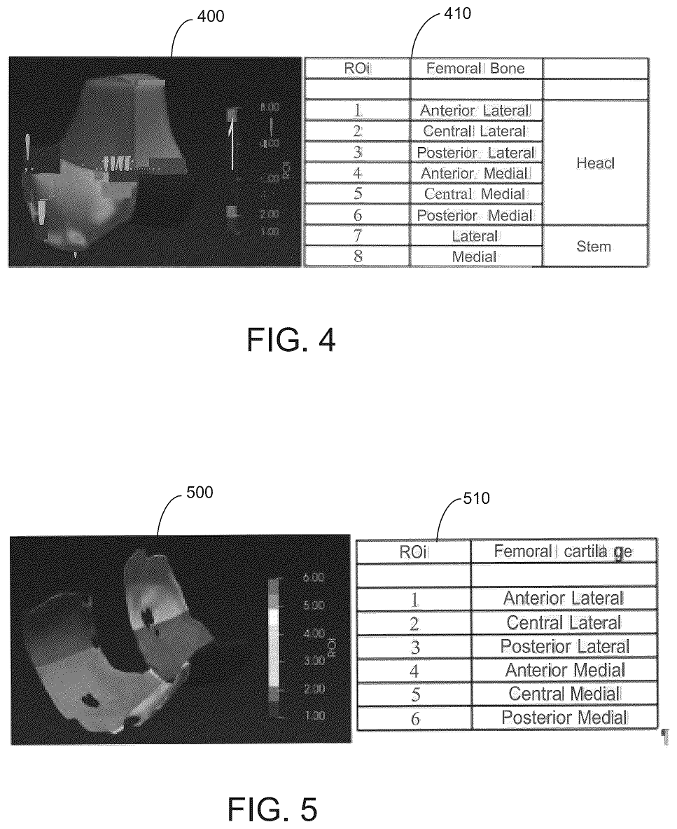

FIG. 4 shows an image 400 illustrating the ROIs of the femur based on ROIs determined by the compute node 130 as described above with respect to FIG. 3. The table 410 shows the names associated with the different ROIs indicated in the image 400.

FIG. 5 shows an image 500 illustrating the ROIs associated with a femoral cartilage. Since the femoral cartilage may be disposed substantially next to the femoral head, the ROIs associated with the femoral head may also be used to define the ROIs of the femoral cartilage. The table 510 shows the names associated with the different ROIs indicated in the image 500.

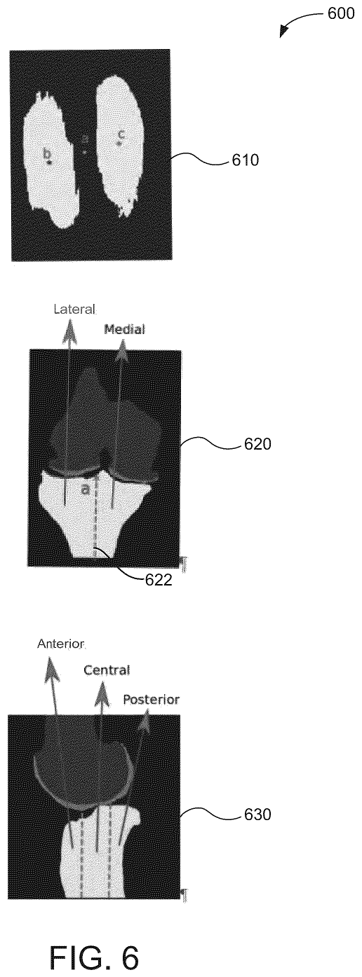

FIG. 6 shows segmented 3D images 600 that may be used by the compute node 130 of FIG. 1 to determine ROIs associated with a tibial bone. Image 610 shows an axial projection of tibial cartilages. The compute node 130, through computer aided analysis, may determine centroid “b” of the lateral cartilage and centroid “c” of the medial cartilage. Next, the compute node 130 may determine midpoint “a” between centroids “b” and “c”.

Image 620 shows a line 622 representing a surface that is projected superior and inferior through the midpoint “a” to divide the tibia into lateral and medial portions.

Next, the compute node 130 determines anterior, central, and posterior portions of the tibia. Image 630 shows a sagittal view of the tibia divided into relatively equal thirds. Each of the thirds is one of the anterior, central, and posterior portions.

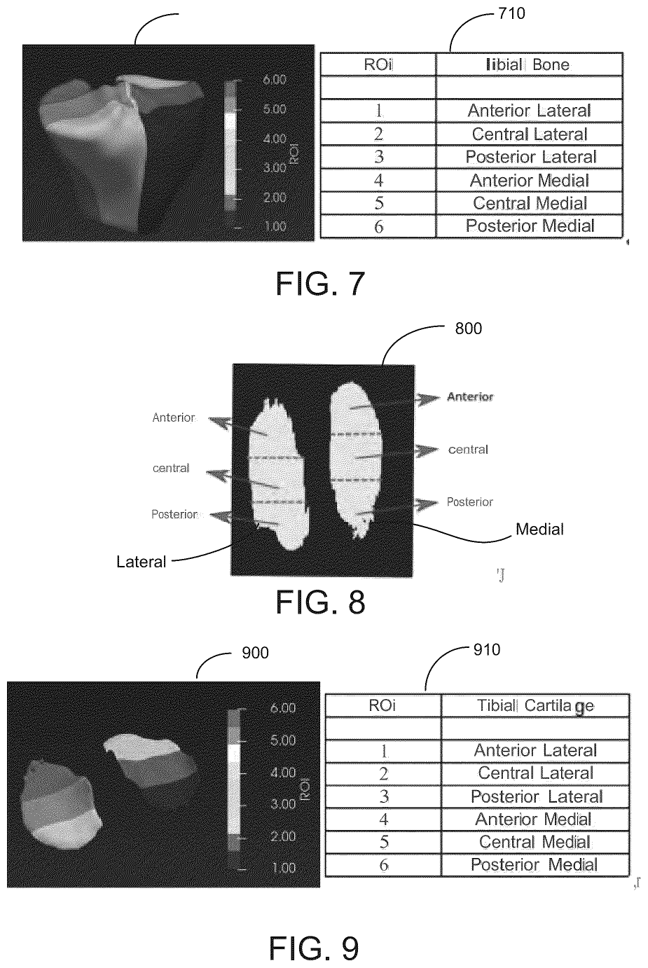

FIG. 7 shows an image 700 illustrating the ROIs associated with the tibia based on the ROIs described above with respect to FIG. 6. Table 710 shows the names associated with the different ROIs indicated in the image 700.

FIG. 8 shows an image 800 that may be used by the compute node 130 of FIG. 1 to determine ROIs associated with tibial cartilage. Image 800 shows an axial view of the tibial cartilage. The compute node 130, through computer aided analysis, may identify medial and lateral portions of the tibial cartilage. Next, the compute node 130 may divide each of the medial and lateral portions into relatively equal thirds. Note that the medial portion may be treated completely independently from the lateral portion. Thus, equal thirds of the medial portion may be different than the equal thirds of the lateral portion. The thirds may be referred to as anterior, central, and posterior regions. An example of the ROIs of the tibial cartilage is shown in image 800.

FIG. 9 shows an image 900 illustrating the ROIs associated with the tibial cartilage as described above with respect to FIG. 8. Table 910 shows the names associated with the different ROIs indicated in the image 900.

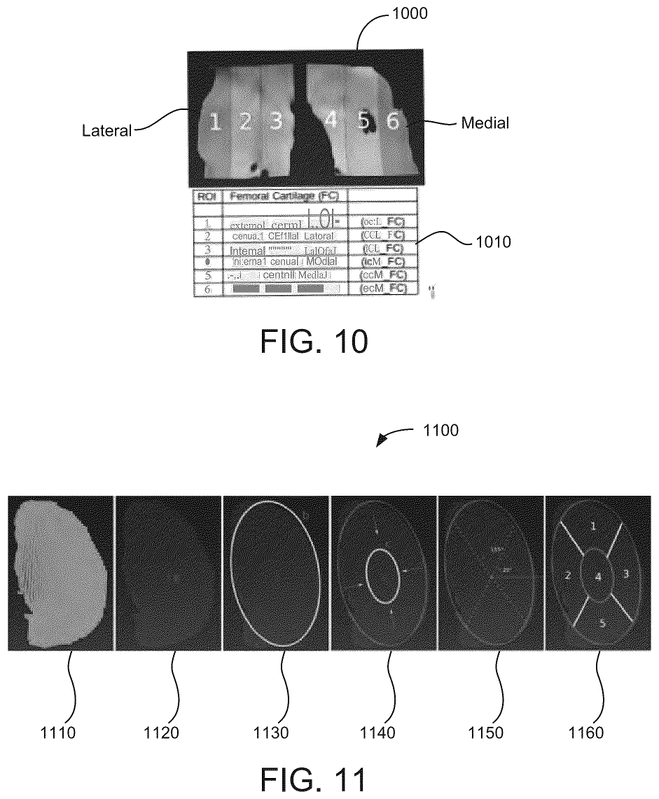

FIG. 10 shows an image 1000 illustrating the ROIs associated with loading regions of the femoral cartilage. The femoral cartilage may be the same as discussed with respect to FIG. 5. Thus, the image 1000 begins with the same femoral cartilage region of FIG. 5. The compute node 130, through computer aided analysis, may divide a central band of the femoral cartilage region into lateral and medial portions. Next, the compute node 130 may divide each of the central medial and central lateral portions into relatively equal thirds. Note that the medial portion may be treated completely independently from the lateral portion. Thus, the equal thirds of the medial portion may be divided differently than the equal thirds of the lateral portion. As a result, the loading-based femoral cartilage is divided into six ROIs as shown. The table 1010 shows the names associated with the different ROIs indicated in the image 1000.

FIG. 11 shows a series of images 1100 that may be used by the compute node 130 of FIG. 1 to determine ROIs associated with loading regions in the tibial cartilage. Referring back to FIGS. 8 and 9, the tibial cartilage may be divided into medial and lateral portions. The compute node 130 may treat the medial and lateral portions separately to determine the related ROIs. Image 1110 shows an axial view of the lateral portion of the tibial cartilage. The compute node 130 may determine a centroid “a” of the lateral portion as shown in image 1120. Next, as shown in image 1130, the compute node 130 projects an ellipse “b” around the portion of lateral portion of the tibial cartilage. Next, the compute node 130 shrinks the ellipse “b” by 80% generating an ellipse “c” as shown in image 1140. Next, the compute node 130 projects lines at approximately 20, 155, 225, and 315 degrees radiating from the centroid “a” as shown in image 1150. Using the ellipse “b” and the lines shown in image 1150, the compute node 130 determines the lateral ROIs shown in image 1160. The compute node 130 may perform similar steps to the medial portion and determine medial ROIs.

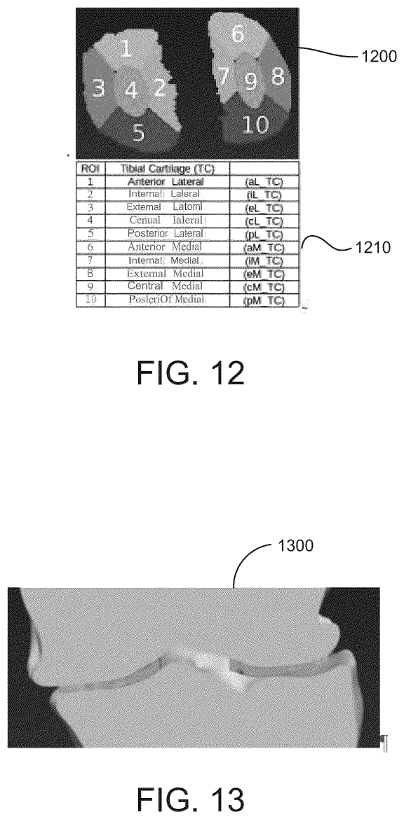

FIG. 12 shows an image 1200 showing the ROIs associated with a loaded tibial cartilage. Table 1210 shows the names associated with the different ROIs indicated in the image 1200.

The 3D visualization of segmented tissue, ROIs, diagnostic images may include a mesh representation. The process of quantification (i.e., quantitative joint analysis and/or diagnosis) or segmentation (i.e., the CNN (Convoluted Neural Network) segmentation model, ROI model) is performed under the domain of the image. The meshing process may be considered a post-processing procedure, in which data (e.g., thickness maps, distance maps, ROI definition) are interpolated once measured. For example, the output of the CNN segmentation model may return a binary segmentation image (where “0” corresponds to the background, and “1” corresponds to the segmented tissue) on which an automatic meshing process is performed. In some examples, tetrahedral elements may be considered in this process. Ultimately, meshing any structure of interest may enable a direct visualization of the rendered volume.

### Quantitative Joint Analysis and Diagnosis

Using the MRI data 150, the segmented 3D images 215, and the corresponding ROIs, selected joints and surrounding tissues can be analyzed and diagnosed. In some cases, the compute node 130 may determine quantitative measurements associated with the selected joint and surrounding tissues using the defined ROIs as part of the analysis and diagnosis. For example, volumes of one or more tissues or bones may be determined corresponding to one or more ROIs. The cubic millimeters (e.g., volume) associated with the selected joints and tissues may be determined by multiplying each voxel within each ROI by the associated dimensions of the voxel. An example of calculated volumes for the femoral bone is shown below in Table 1. Similar volumes may be calculated for other bones and tissues.

TABLE 1

| | | | Tissue Volume [mm3] | | | |
|---|---|---|---|---|---|---|
| Region of Interest (ROI) | | ID | FB | FC | TB | TC |
| Anterior Medial | (AM) | 1 | 23540.59 | 3197.57 | 22991.53 | 458.06 |
| Central Medial | (CM) | 2 | 30915.97 | 3323.00 | 31622.64 | 630.47 |
| Posterior Medial | (PM) | 3 | 22427.22 | 1938.03 | 14144.56 | 422.24 |
| Anterior Lateral | (AL) | 4 | 18486.12 | 1158.31 | 16045.65 | 601.16 |
| Central Lateral | (CL) | 5 | 34306.24 | 2929.23 | 30995.43 | 905.60 |
| Posterior Lateral | (PL) | 6 | 20747.11 | 2063.27 | 13037.89 | 363.43 |
| FB Stem Medial | (FB_SM) | 7 | 27856.93 | | | |
| FB Stem Lateral | (FB_SL) | 8 | 16923.06 | | | |

In a similar manner, a surface area, in square millimeters, may be calculated. For example, using edge filtering techniques, edge contour information may be determined. Voxel dimensions may be used with the edge contour information and ROI information to determine the surface area of each ROI.

Joint space width may also be determined. For example, cartilage within an ROI may be identified. Then, the cartilage is “projected” toward the bones of the joint until the surface of the bone is detected. This action determines upper and lower boundaries of the joint space. The distances associated with the joint space may be determined (measured) based on voxel dimensions. Distances between the bones may be measured at several positions within the joint. In some examples, a distribution of measurements may be determined and the joint space width may be defined as the mean of the lowest five percent of the measurements within the distribution. Furthermore, the joint space widths may be determined with respect to the ROIs associated with the joint. An example of calculated joint space widths is shown below in Table 2. In some examples, joint space narrowing may be determined by comparing the joint space width of a selected joint determined for two or more different measurement times. Joint space narrowing may be used to diagnose and/or predict disease progression. The quantitative measurements associated with joint space width may be mapped to a 3D image. FIG. 13 shows an image 1300 showing a 3D joint space width image based on the joint space determined from the voxel data. Thus, the image 1300 may be a diagnostic 3D image (e.g., an example diagnostic 3D image 240 as described with respect to FIG. 2).

TABLE 2

| Articular Space ROI | Joint Space Width (JSW) [mm] |
|---|---|
| Medial | 2.71 |
| Lateral | 2.15 |

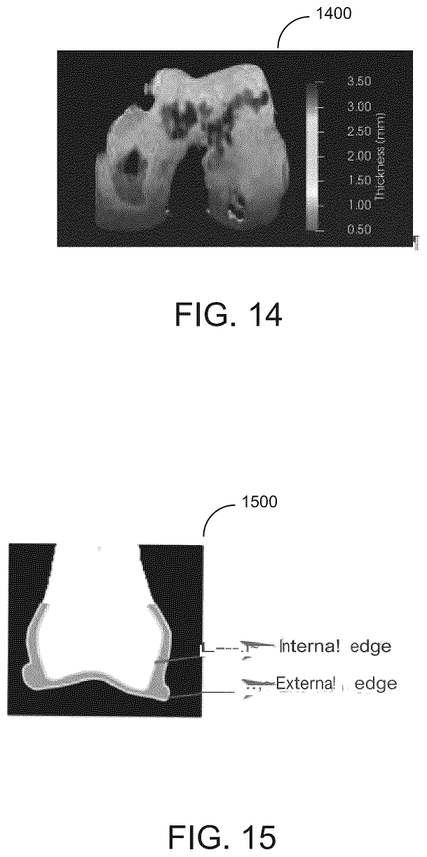

Additional diagnostic 3D images may be generated and displayed based on other determined quantitative data. For example, the compute node 130 may determine one or more thickness maps of cartilage within the selected joint area. To generate the thickness map, the compute node 130 may examine a slice of a 2D cartilage segmentation. The cartilage boundary may be extracted, and a “skeleton” associated with the cartilage may be extracted. The skeleton may be a skeletal representation of the cartilage that may be based on reducing intensity of foreground areas in the cartilage segmented image. The skeletal representation preserves connectivity of the cartilage image. Using the extracted cartilage boundary and the skeletal representation, the thickness of the cartilage, within the current 2D slice may be determined. This procedure may be repeated for all of the slices in the cartilage volume to generate the cartilage thickness 3D map. FIG. 14 shows an example femoral cartilage thickness map 1400 based on the extracted cartilage boundary and the skeletal representation, as described above.

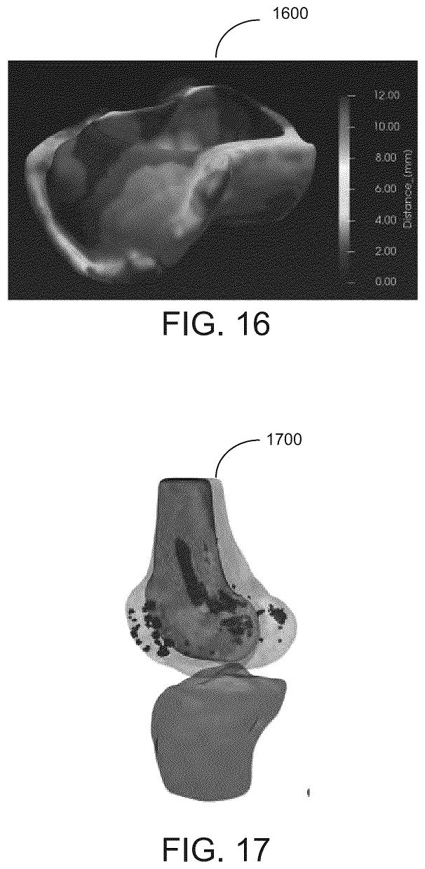

In another example, a 3D surface distance map may be generated by the compute node 130 using one or more diagnostic 3D images 240. The distance surface maps may be computed based on any two arbitrary surfaces. FIG. 15 shows an image 1500 with two arbitrary surfaces. The two arbitrary surfaces may include a first surface (e.g., internal edge) and a second surface (e.g., external edge). A distance function may estimate distances between each surface to generate a related 3D surface distance map. FIG. 16 shows an image 1600 depicting a surface distance map (e.g., a thickness map) associated with the two surfaces shown in FIG. 15.

In some examples, a surface topology map may be determined from the thickness map (such as the thickness map of FIG. 16) and/or distance information. The topology map may describe a roughness/smoothness or homogeneous/heterogeneous of the thickness map. The surface topology map may enable the clinician to diagnose bone and subchondral bone perforations, lacerations, or lesions.

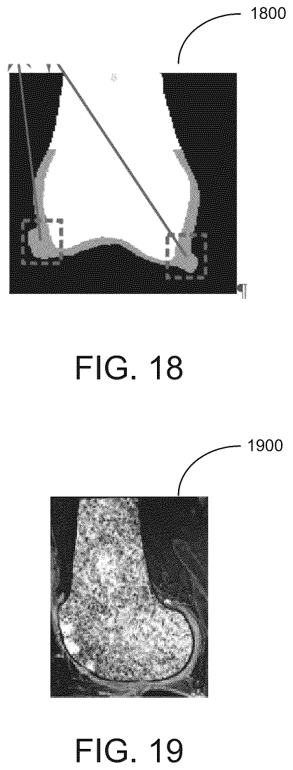

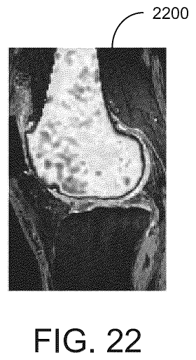

Bone and subchondral bone inflammation may be determined based at least on the texture-pattern analysis that correlates with a determined water concentration. (Determining water concentration is discussed in more detail below in conjunction with FIG. 22.) In some examples, the bone and subchondral bone inflammation may be associated with one or more ROIs (loading-based ROIs, subchondral bone ROI, femoral bone ROIs, and the like). An inflamed region may be identified, and an associated volumetric measurement may be determined. FIG. 17 shows an image 1700 depicting bone edema/inflammation. Furthermore, Osteophytes (bone spurs) may be identified and displayed. The Osteophytes may be determined through surface analysis of one or more diagnostic 3D images 240. FIG. 18 shows an example image 1800 showing bone spurs 1810.

Image texture analysis may be performed to further identify and diagnose other aspects associated with the selected joint. Texture analysis may be based on a distribution of voxel intensities (so-called first-order metrics) and voxel relationships (so-called higher-order metrics) to determine statistical trends which may underlie joint damage or disease. First-order metrics may determine a spatial distribution of gray level intensities. For example, first-order metrics may describe a homogeneity/uniformity or heterogeneity/randomness of voxel intensities. Higher-order metrics may describe inter-voxel relationships such as gray level co-occurrence which describes how frequently pairs of voxels with a specific intensity appear within an ROI.

A first example of a first-order metric is an Entropy metric. In this context, Entropy is a measure of uncertainty or randomness of intensity values within an image, or within an ROI of an image. Entropy may be described by the following equation:

$$entrophy = - {{\sum }_{i = 1}}^{{N}_{g}} p ( i ) {log}_{2} ( p ( i ) + e )$$

Where Ng is the number of intensity levels, p(i) is the normalized first-order histogram and e is an arbitrary small positive scalar (e.g., 2.2E-16). The first order histogram is given by the voxel frequency as a function of the voxel intensity. If the first order histogram is divided by the total number of voxels inside the region, the normalized first order histogram is obtained. FIG. 19 shows an example Entropy image 1900 where lighter areas indicate higher levels of randomness.

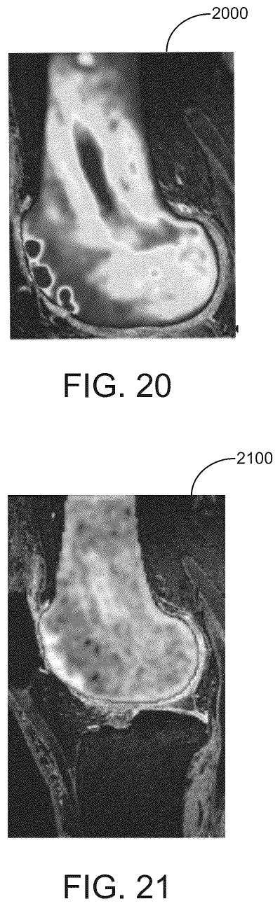

Another example of a first-order metric is an energy metric. One example of the energy metric may be described by the following equation:

$$energy = {{\sum }_{i = 1}}^{{N}_{g}} {( X ( i ) + c )}^{2}$$

where Np is the number of voxels within an ROI, X(i) is voxel intensity and c is an optional offset to avoid negative values of X(i). FIG. 20 shows an example energy image 2000 inside a femoral bone. Brighter values indicate a higher energy.

One example of a higher-order metric is a gray level co-occurrence matrix (GLCM). In general, a co-occurrence matrix is given by the frequency that a given combination of intensities appear within a region. If the co-occurrence matrix is divided by the total number of occurrences, the normalized co-occurrence matrix is obtained. A GLCM of size Ng×Ng may be interpreted as a second-order joint probability function of an ROI. In some embodiments, the joint probability function may describe probabilities that given combinations of intensities appear within a region. A GLCM joint entropy may be described by the following equation:

$$joint entrophy = - {{\sum }_{i = 1}}^{{N}_{g}} {{\sum }_{j = 1}}^{{N}_{g}} p ( i , j ) {log}_{2} ( p ( i , j ) + e )$$

where p(i,j) is a normalized co-occurrence matrix, Ng is the number of discrete intensity levels in the ROI, and e is an arbitrarily small positive scalar (e.g., 2.2E-16). FIG. 21 shows an image 2100 depicting joint entropy within a femoral bone.

Another example of a higher-order metric is a GLCM Inverse Difference (ID). This metric is a measure of homogeneity inside a ROI. In some examples, more uniform gray levels in the image will result in higher overall GLCM ID values. The GLCM ID may be described by the equations:

$$ID = \frac{{{\sum }_{k = 0}}^{{N}_{g} - 1} {p}_{x - y} ( k )}{1 + k}$$

$$and$$

$${p}_{x - y} ( k ) = {{\sum }_{i = 1}}^{{N}_{g}} {{\sum }_{j = 1}}^{{N}_{g}} p ( i , j )$$

where |i−j|=k, and k=0, 1, . . . , Ng−1. FIG. 22 shows an image 2200 depicting a GLCM ID of a femoral bone. Note that lighter values indicate a higher homogeneity.

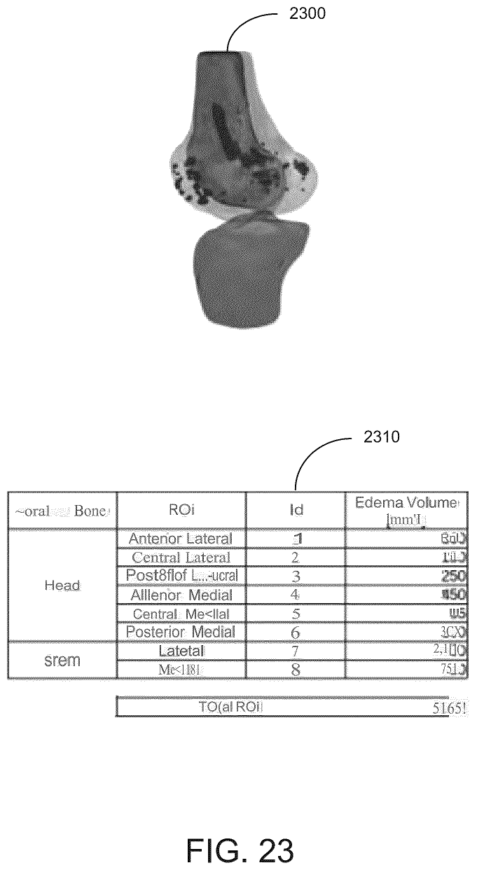

One example of a diagnosis that uses first-order and higher-order metrics is to identify regions associated with bone inflammation, such as bone edema. Bone edema may be indicated by a build-up of fluid within the bone. In one example, to diagnose bone edema, an ROI is selected. The ROI may be any feasible bone, or portion of bone, such as the femur. The energy of the ROI is estimated by, for example, the energy equation described above. The energy information may then be interpolated to a previously defined diagnostic 3D image, placing the energy information in a 3D representation. Next, edema volumes within the ROI may be determined by comparing the energy information to a predetermined threshold. FIG. 23 shows a diagnostic 3D image 2300 depicting detected edema. In some examples, tabulated data corresponding to the edema volumes may be presented to the clinician or user as shown in table 2310. Thus, in some cases, water concentration may be indicated by the detected energy associated with one or more bones.

In some examples, any of the segmented 3D images and diagnostic 3D images described herein may be made available to clinicians and/or users to aid in the diagnosis related to the selected joint. In some cases, a dashboard may be presented to allow the clinician or user to select any particular image as well as interact with the selected image in real time.

In some variations, the compute node 130 may provide statistical analysis based on the quantitative measurements that have been determined. For example, the compute node 130 can compare the patient's quantitative measurements to mean population values to determine if the patient's measurements are statistical outliers. In addition, the compute node 130 may compare a patient's demographic data with aggregated demographic data. The patient's demographic data may be based on the complementary patient data 112 as described with respect to FIG. 1. This statistical analysis may assist in diagnosing joint health or disease.

In some embodiments, the compute node 130 may identify and classify joint lesions within one or more ROIs. The joint lesions may be identified and/or classified based on a comparison of the patient's quantitative measurements to thresholds. For example, a patient's tissue thickness may be compared to thickness thresholds. If the tissue thickness is less than the thickness threshold, the compute node 130 may identify the ROI as including a joint lesion and, further, may quantify the lesion size and shape. In some cases, the compute node 130 may classify or estimate damage associated with the lesion. The estimation of the damage may include MOAKS or/and ICRS. (WOMAC indicates degree of pain, which is not obtained from the diagnosis pipeline and WORMS is an old version of MOAKS The identification and classification of joint lesions may advantageously provide insight to clinicians for a diagnosis of osteoarthritis.

In some variations, the compute node 130 may predict a patient's progress with respect to one or more joint-related conditions, including osteoarthritis. The prediction (via a deep-learning classification model) may be based on a trained convolutional neural network that may accept current and previous quantitative measurement data as well as current and previous complementary patient data 112. The quantitative measurement data may be provided directly or indirectly by any of the processing steps described above with respect to the determination and/or generation of 3D diagnostic images as described in FIGS. 13-23. The output of the deep-learning model (sometimes referred to as a patient's prognosis or future patient's status in general) can be interpreted as a discrete probability distribution that can take values on a countable number of possible output classes. In one example, the options may be four different classes: pain and joint space width (JSW) progression, pain progression, JSW progression or neither pain nor JSW progression.

For example, the compute node 130 may use MRI images 150, quantitative data determined from the 3D diagnostic images (such as any images from FIGS. 13-23), and complementary patient data 112 as inputs to a predictive model. The predictive model may include a deep-learning model executed by a trained convolutional neural network, which generates the first and most relevant output of the prognosis algorithm, given by the class prediction. Furthermore, the complete prognosis algorithm may also include one or more deterministic algorithms to quantify the importance of relevant features (e.g., lesions) and regions (e.g., ROIs) to determine a patient's prognosis.

In a typical deep-learning model, prepared or designed to solve image classification tasks, the input image is first passed through a series of sequential filtering steps, which may be referred to as convolutional layers. Note that at each convolutional layer, several filters can be applied in parallel. After the application of each filter a new matrix may be obtained. These matrices may be called feature activation maps (also referred to as activation maps or feature maps). The stacked set of features maps may function as the input for the next convolutional layer. This block of operations may be referred to as the perceptual block. Note that the matrix components that define the filtering matrices may be learned during the training process and may be called weights. In general there can be several convolutional layers before proceeding to a subsequent step of the deep-learning model, which may be called the logical block. After the input image is sequentially filtered by the elements of the perceptual block, the final activation map may be reshaped as a 1D vector. At this step, the tabulated complementary data can be added to the 1D vector by a concatenation operation. The resulting 1D vector is the input for a subsequent series of operations of the deep-learning model. Typically, this vector is passed through a series of dense layers that incorporate non-linear operations between the 1D vector components. This process ends with a final layer that contains N neurons, one for each class, that record values that can be interpreted as a discrete probability distribution. The final decision of the network is usually defined as the neuron/class with the highest probability. The deep-learning model is able to learn the correct classes by a supervised training procedure, where known annotated data is shown to the network. Afterwards, the ability of the network to make correct predictions could be tested on unseen data.

In addition to the classification output, the compute node 130 also may produce and/or provide additional information in order to enrich the level of explanation of the obtained result. For example, the compute node 130 may quantify the importance of some regions of the input image based on their relevance for the decision taken by the model. More specifically, the method used for this task may include the determination (e.g., generation) of attention maps, which work as follows. If the classification output is given by “y” and the feature activation maps for a given convolutional layer are given by “A_k_ij”, where k is the index counting the number of filters at the given convolutional layer step and i and j are indexes that cover the width and height of each feature map in pixels, then the importance of a given feature activation is quantified by the gradient of “y” with respect to “A_k_ij”, which can be expressed by “dy/dA_k_ij”. In practice, the gradient may be obtained by backpropagation, following the flow of evaluations from the output neuron (described above) to the corresponding activation maps. Based on these gradient values, it may be possible to compute the activation map weight a_k, which is a single number for each map k, obtained from the global average pooling of the matrix dy/dA_k_ij. A final attention map is then computed by the weighted sum of a_k times A_k, thus the dimensions of the map have the same dimensions of the feature maps in pixel units. Since the output of convolutional layers (feature activation maps) are smaller than the input image, the attention map is indeed a coarse representation of the original input image resolution.

In another example, regarding the generation of extra information to support the decision of the classification model, the compute node 130 may perform principal component analysis to determine the importance of particular features for the patient prognosis. For example, the compute node 130 may compute the principal components (PCs) associated with the full sample of input vectors (tabulated data or full 1D vector including feature maps information from the convolutional layers). In order to understand the properties of the PCs and how they can be used to quantify the importance of a given feature, it is convenient to recall how they are computed. Consider a training sample with n_p patients, where each single patient has an input vector with n_f features. From this data it is possible for the compute node 130 to compute an input data matrix X, with shape [n_p, n_f], which is further normalized and standardized (z-scored) In this context, z-scored means that the contents of the matrix X may now represent deviations with respect to the mean in standard deviation units). From X, we can compute the covariance matrix C_x=1/n_p X′X, with X′ the transpose of X. In this case, the PCs are defined as the eigenvectors of C_x. These vectors may determine the direction of higher variance in the space of input vectors, and we can have as many n_features of these vectors.

By construction, the PC vectors indicate the directions in the space of features of major variance of the input data, therefore these vectors may be correlated to the directions along which we see a more rapid change in the class of the patients. In one example, the main PC may be given by a vector that points along the direction of the age axis. This means that as one moves along the age of patients, one may find a rapid variation of the classes of patients, from patients that show non-progression of pain to those that do show progression. Thus, one can conclude that age is an important feature to determine the class of a given patient. Furthermore, the hierarchy between PC vectors as a function of the variance is given by the values of the corresponding eigenvalues, thus the compute node 130 can order the PC vectors by relevance in terms of data variance. Finally, since the alignment between the input feature vectors and the PC directions may be given by a pairwise dot product between them, the compute node 130 can conclude that the most relevant features of the input data, that determine the differences along the axis defined by a given PC vector, are those that coincide with the position of the PC vector component of higher value.

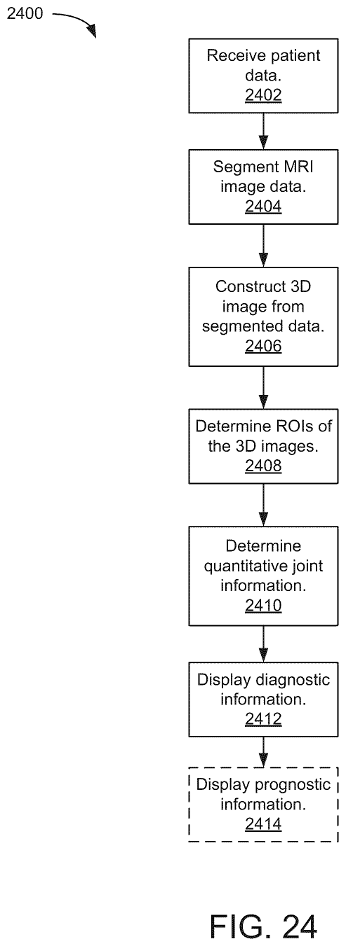

FIG. 24 is a flowchart depicting an example of one method 2400 for determining quantitative joint tissue degeneration measurements, in accordance with some embodiments. Some examples may perform the operations described herein with additional operations, fewer operations, operations in a different order, operations in parallel, and some operations differently. The method 2400 is described as being performed by the compute node 130 of FIG. 1 for convenience. In other embodiments, the method 2400 may be performed by any feasible computer, processor, server, cloud compute resource, or the like.

The method 2400 may begin as the compute node receives patient data in block 2402. The patient data may include MRI image data 150 and complementary patient data 112 as described with respect to FIG. 1.

Next, in block 2404 the compute node 130 may segment the MRI image data 150. For example, as described with respect to FIG. 2, the compute node 130 may perform a neural network processing procedure 210 to discriminate between different joint tissues and also determine boundaries associated with joints and joint tissues. The neural network processing procedure 210 may generate a number of images, referred to as segmented images. In some variations, the compute node 130 may also remove artifacts from the segmented images. For example, the compute node 130 may provide seven segmented 2D image sets including a femoral bone image, a femoral cartilage image, a tibial bone image, a tibial cartilage image, a patellar bone image, a patellar cartilage image, and a background image. In other examples, the images may include other bone, joints, and/or tissues.

Next, in block 2406 the compute node 130 may construct (mesh) 3D images from the segmented MRI data. For example, the compute node 130 may mesh together the 2D image sets to form related volumetric 3D images.

Next, in block 2408, the compute node 130 may determine one or more ROIs of the 3D images. For example, the ROIs may be determined by any of the operations described with respect to FIGS. 3-12.

Next, in block 2410, the compute node 130 may determine quantitative joint information based, at least in part, on the determined ROIs and the meshed 3D images. For example, as described with respect to FIGS. 13-16, the compute node 130 may determine bone, tissue, and/or joint measurements, including tissue volume, bone volume, cartilage thickness, bone surface area, and the like. In some examples, the compute node 130 may determine diagnostic 3D images based on the quantitative joint information. Example diagnostic 3D images may include renderings of determined bones, tissues, related volumes, cartilage thickness, bone surface areas, edemas, bone spurs and the like as described herein with respect to FIGS. 13-23. The diagnostic 3D images may be used to determine joint diagnosis and prognosis.

Next, in block 2412, diagnostic information based at least in part on the determined quantitative joint information may be displayed. For example, the diagnostic 3D images and/or quantitative joint information may be displayed to a clinician or user. The displayed information may be used to determine or diagnose a body joint.

Next, in block 2414, prognostic information may be displayed. In this optional step (denoted in FIG. 24 with dashed lines), prognostic information associated with the patient may be displayed. For example, a patient's joint degeneration may be predicted based on the determined quantitative joint information and the complementary patient information.

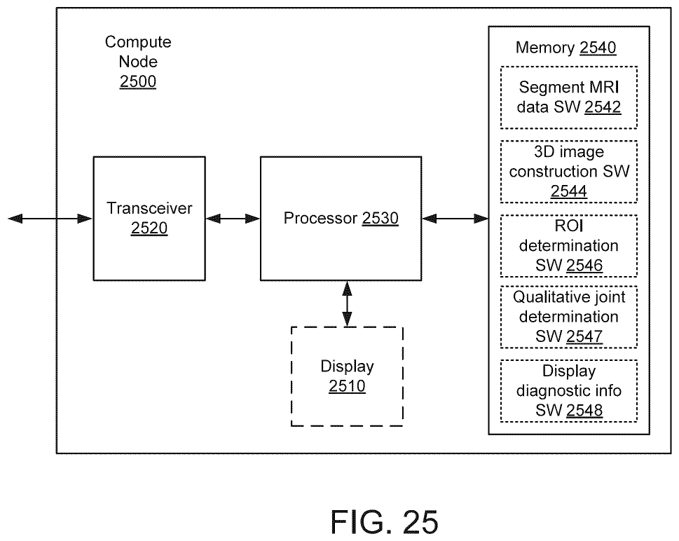

FIG. 25 shows a block diagram of a compute node 2500 that may be an embodiment of the compute node 130 of FIG. 1. The compute node 2500 may include a display 2510, transceiver 2520, a processor 2530, and a memory 2540. The transceiver 2520, which may be coupled to a network (not shown), may transmit signals to and receive signals from other wired or wireless devices. Although not shown for simplicity, a transceiver controller may be implemented within the processor 2530 and/or the memory 2540 to control transmit and receive operations of the transceiver 2520 including, for example, receiving MRI data and transmitting 3D images and quantitative joint data.

The display 2510, which is coupled to the processor 2530 may be optional, as shown by dashed lines in FIG. 25. The display 2510 may be used to display meshed 3D images, diagnostic 3D images, quantitative joint data, or any other feasible images or data.

The processor 2530, which is also coupled to the transceiver 2520 and the memory 2540, may be any one or more suitable processors capable of executing scripts or instructions of one or more software programs stored in the compute node 2500 (such as within memory 2540).

The memory 2540 may include a non-transitory computer-readable storage medium (e.g., one or more nonvolatile memory elements, such as EPROM, EEPROM, Flash memory, a hard drive, etc.) that may store the following software modules:
a segment MRI data software (SW) module 2542 to segment MRI data;
a 3D image construction SW module 2544 to construct (e.g., mesh) 3D images from segmented MRI data;
a ROI determination SW module 2546 to determine one or more ROIs within constructed 3D images;
a quantitative joint information determination SW module 2547 to determine quantitative joint information from the meshed 3D images and ROIs;
a display diagnostic information SW module 2548 to display determined diagnostic information; and
a display prognostic information SW module 2549 to display determined prognostic information.
   Each software module includes program instructions that, when executed by the processor 2530, may cause the compute node 2500 to perform the corresponding function(s). Thus, the non-transitory computer-readable storage medium of memory 2540 may include instructions for performing all or a portion of the operations described herein.

The processor 2530, which is coupled to the transceiver 2520, and the memory 2540, may be any one or more suitable processors capable of executing scripts or instructions of one or more software programs stored in the compute node 2500 (e.g., within the memory 2540).

The processor 2530 may execute the segment MRI data SW module 2542 to receive MRI image data and generate segmented MRI data, for example, as described with respect to FIGS. 1 and 2. In some examples, the processor 2530 may execute a deep-learning based algorithm to generate the segmented MRI images/data.

The processor 2530 may execute the 3D image construction SW module 2544 to generate 3D images. In some examples, the processor 2530 may mesh together one or more segmented MRI images and may also remove any detected artifacts.

The processor 2530 may execute the ROI determination SW module 2546 to autonomously determine one or more ROIs that may be associated with bones, cartilage, cartilage loading areas, or the like as described with respect to FIGS. 3-12.

The processor 2530 may execute the quantitative joint information determination SW module 2547 to determine quantitative joint information. The quantitative joint information may be based on the ROIs determined by execution of the ROI determination SW module 2546. For example, the processor 2530 may determine energy, entropy, as well as compute cartilage thickness or any other feasible joint information as described herein. For example, quantitative joint information may be determined, and related images determined as described with respect to FIGS. 13-23.

The processor 2530 may execute the display diagnostic information SW module 2548 to display any diagnostic feasible images and/or data. For example, the processor 2530 may render segmented 3D images and/or diagnostic 3D images based on determined ROIs or other information. The processor 2530 may cause the images or data to be displayed on the display 2510 or transmitted through a network and displayed on any feasible device.

The processor 2530 may execute the display prognostic information SW module 2549 to display any feasible prognostic images and/or data. For example, the processor 2530 may display attention maps, associated quantitative data on the display 2510 or any other feasible device.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of determining joint tissue degeneration, the method comprising:
receiving magnetic resonance imaging (MRI) data for a selected joint;
generating MRI segments based at least in part on the MRI data, wherein the MRI segments are two-dimensional probability maps;
generating three-dimensional models based at least in part on the MRI segments;
autonomously determining one or more regions of interest (ROIs) based at least in part on the three-dimensional models;
generating three-dimensional diagnostic images illustrating selected tissue degeneration areas based at least in part on the three-dimensional models and the one or more ROIs; and
displaying the three-dimensional diagnostic images.

2. The method of claim 1, wherein the step of generating MRI segments includes processing MRI images with a neural network to discriminate between different joint tissues, determine boundaries of each of the joint tissues and generating segmented images.

3. The method of claim 2, wherein after processing MRI images with a neural network an upsampling algorithm is used, which includes voxel isotropication, image alignment and a multi-planar combination model; the upsampling algorithm allows to combine complementary information from different anatomical views to provide high resolution 3D representations of the joint.

4. The method of claim 3, wherein after applying the upsampling algorithm a statistical shape modeling is used to automatically select the side of the input knee sequence.

5. The method of claim 1, wherein the one or more ROIs are based at least in part on topological gradients of the three-dimensional models.

6. The method of claim 5, wherein the topological gradients are identified based on computer aided analysis of the three-dimensional models.

7. The method of claim 1, wherein the one or more ROIs include three-dimensional bone regions near the selected joint.

8. The method of claim 7, wherein the three-dimensional bone regions include a femur, a tibia, or a combination thereof.

9. The method of claim 1, wherein the one or more ROIs include three-dimensional cartilage regions near the selected joint.

10. The method of claim 9, wherein the three-dimensional cartilage regions include a femoral cartilage region, a tibial cartilage region, a tibial cartilage loading region, or a combination thereof.

11. The method of claim 1, wherein the three-dimension diagnostic images include a three-dimensional thickness map of a joint space associated with the selected joint.

12. The method of claim 11, wherein determining the three-dimensional thickness map comprises:
estimating an edge of one or more cartilage regions within an MRI segment associated with the selected joint;
determining a skeleton associated with the selected joint;
determining a volume based on the estimated edge and skeleton; and
determining the thickness associated with the joint based on the volume, summed over the MRI segment.

13. The method of claim 1, wherein the three-dimensional diagnostic images include a bone edema and inflammation image.

14. The method of claim 13, wherein the bone edema and inflammation image is based at least in part on determining a water concentration in one or more tissues associated with the selected joint.

15. The method of claim 1, wherein the three-dimensional diagnostic images include a joint space width image.

16. The method of claim 15, further comprising determining a mean value from a lowest five percent distribution of joint spaces.

17. The method of claim 1, wherein the three-dimensional diagnostic images include a bone spur identification image.

18. The method of claim 1, further comprising determining a water concentration of bones and cartilage associated with the select joint based at least in part on determining a uniformity of voxel intensity.

19. The method of claim 18, wherein determining the uniformity includes determining an entropy associated with one or more three-dimensional models.

20. The method of claim 18, wherein determining the uniformity includes determining an energy associated with voxels of one or more three-dimensional models.

21. The method of claim 18, wherein determining the uniformity includes determining a gray level co-occurrence matrix of joint entropy.

22. The method of claim 18, wherein determining the uniformity includes determining a gray level co-occurrence matrix of inverse difference.

23. The method of claim 1, further comprising:
determining quantitative joint information based at least in part on the three-dimensional models; and
displaying the quantitative joint information.

24. The method of claim 1, further comprising:
predicting joint-related conditions based at least in part on the three-dimensional diagnostic images; and
displaying an image showing, at least in part, the predicted joint-related conditions.

25. The method of claim 24, wherein the predicting includes determining a classification of the predicted joint-related conditions.

26. The method of claim 25, wherein the classifications include pain progression, joint space width progression, pain and joint space width progression, neither pain nor joint space width progression, or a combination thereof.

27. The method of claim 24, wherein the predicting is based on a deep-learning model executed by a trained convolutional neural network.

28. A system for determining joint tissue degeneration, comprising:
one or more processors; and
a memory configured to store instructions that, when executed by the one or more processors, cause the system to:
receive magnetic resonance imaging (MRI) data for a selected joint;
generate MRI segments based at least in part on the MRI data, wherein the MRI segments are two-dimensional probability maps;
generate three-dimensional models based at least in part on the MRI segments;
autonomously determine one or more regions of interest (ROIs) based at least in part on the three-dimensional models;
generate three-dimensional diagnostic images illustrating selected tissue degeneration areas based at least in part on the three-dimensional models and the one or more ROIs; and
display the three-dimensional diagnostic images.

29. The system of claim 28, wherein to generate MRI segments the system is configured to process MRI images with a neural network to discriminate between different joint tissues, determine boundaries of each of the joint tissues and generating segmented images.

30. The system of claim 29, wherein after processing MRI images with a neural network an upsampling algorithm is used, which includes voxel isotropication, image alignment and a multi-planar combination model; the upsampling algorithm allows to combine complementary information from different anatomical views to provide high resolution 3D representations of the joint.

31. The system of claim 30, wherein a statistical shape modeling is used after using the upsampling algorithm to automatically select the side of the input knee sequence.

32. The system of claim 28, wherein the one or more ROIs are based at least in part on topological gradients of the three-dimensional models.

33. The system of claim 32, wherein the topological gradients are identified based on computer aided analysis of the three-dimensional models.

34. The system of claim 28, wherein the one or more ROIs include three-dimensional bone regions near the selected joint.

35. The system of claim 34, wherein the three-dimensional bone regions include a femur, a tibia, or a combination thereof.

36. The system of claim 28, wherein the one or more ROIs include three-dimensional cartilage regions near the selected joint.

37. The system of claim 36, wherein the three-dimensional cartilage regions include a femoral cartilage region, a tibial cartilage region, a tibial cartilage loading region, or a combination thereof.

38. The system of claim 28, wherein the three-dimension diagnostic images include a three-dimensional thickness map of a joint space associated with the selected joint.

39. The system of claim 38, wherein execution of the instructions causes the system to:
estimate an edge of one or more cartilage regions within an MRI segment associated with the selected joint;
determine a skeleton associated with the selected joint;
determine a volume based on the estimated edge and skeleton; and
determine the thickness associated with the joint based on the volume, summed over the MRI segment.

40. The system of claim 28, wherein the three-dimensional diagnostic images include a bone edema and inflammation image.

41. The system of claim 40, wherein the bone edema and inflammation image is based at least in part on a determination of a water concentration in one or more tissues associated with the selected joint.

42. The system of claim 28, wherein the three-dimensional diagnostic images include a joint space width image.

43. The system of claim 42, wherein execution of the instructions causes the system to determine a mean value computed from a lowest five percent distribution of joint spaces.

44. The system of claim 28, wherein the three-dimensional diagnostic images include a bone spur identification image.

45. The system of claim 28, wherein execution of the instructions causes the system to determine a water concentration of bones and cartilage associated with the select joint based at least in part on a determination of uniformity of voxel intensity.

46. The system of claim 45, wherein instructions to determine the water concentration include instructions to determine an entropy associated with one or more three-dimensional models.

47. The system of claim 45, wherein instructions to determine the water concentration include instruction to determine an energy associated with voxels of one or more three-dimensional models.

48. The system of claim 45, wherein instructions to determine the water concentration include instructions to determine a gray level co-occurrence matrix of joint entropy.

49. The system of claim 45, wherein instructions to determine the water concentration include instructions to determine a gray level co-occurrence matrix of inverse difference.

50. The system of claim 28, wherein execution of the instructions causes the system to:
determine quantitative joint information based at least in part on the three-dimensional models; and
display the quantitative joint information.

51. The system of claim 28, wherein execution of the instructions causes the system to:
predict joint-related conditions based at least in part on the three-dimensional diagnostic images; and
display an image showing, at least in part, the predicted joint-related conditions.

52. The system of claim 51, wherein the instructions to predict further include instructions to determine a classification of the predicted joint-related conditions.

53. The system of claim 52, wherein the classifications include pain progression, joint space width progression, pain and joint space width progression, neither pain nor joint space width progression, or a combination thereof.

54. The system of claim 51, wherein the instructions to predict are based on a deep-learning model executed by a trained convolutional neural network.

55. A non-transitory computer-readable storage medium comprising instructions that, when executed by one or more processors of a system, cause the system to perform operations comprising:
receiving magnetic resonance imaging (MRI) data for a selected joint;
generating MRI segments based at least in part on the MRI data, wherein the MRI segments are two-dimensional probability maps;
generating three-dimensional models based at least in part on the MRI segments;
autonomously determining one or more regions of interest (ROIs) based at least in part on the three-dimensional models;
generating three-dimensional diagnostic images illustrating selected tissue degeneration areas based at least in part on the three-dimensional models and the one or more ROIs; and
displaying the three-dimensional diagnostic images.

56. The non-transitory computer-readable storage medium of claim 55, wherein to generate MRI segments the non-transitory computer-readable storage medium is configured to process MRI images with a neural network to discriminate between different joint tissues, determine boundaries of each of the joint tissues and generating segmented images.

57. The non-transitory computer-readable storage medium of claim 56, wherein after processing MRI images with a neural network an upsampling algorithm is used, which includes voxel isotropication, image alignment and a multi-planar combination model; the upsampling algorithm allows to combine complementary information from different anatomical views to provide high resolution 3D representations of the joint.

58. The non-transitory computer-readable storage medium of claim 57, wherein a statistical shape modeling is used after using the upsampling algorithm to automatically select the side of the input knee sequence.

59. The non-transitory computer-readable storage medium of claim 55, wherein the one or more ROIs are based at least in part on topological gradients of the three-dimensional models.

60. The non-transitory computer-readable storage medium of claim 59, wherein the topological gradients are identified based on computer aided analysis of the three-dimensional models.

61. The non-transitory computer-readable storage medium of claim 55, wherein the one or more ROIs include three-dimensional bone regions near the selected joint.

62. The non-transitory computer-readable storage medium of claim 61, wherein the three-dimensional bone regions include a femur, a tibia, or a combination thereof.

63. The non-transitory computer-readable storage medium of claim 55, wherein the one or more ROIs include three-dimensional cartilage regions near the selected joint.

64. The non-transitory computer-readable storage medium of claim 63, wherein the three-dimensional cartilage regions include a femoral cartilage region, a tibial cartilage region, a tibial cartilage loading region, or a combination thereof.

65. The non-transitory computer-readable storage medium of claim 55, wherein the three-dimension diagnostic images include a three-dimensional thickness map of a joint space associated with the selected joint.

66. The non-transitory computer-readable storage medium of claim 65, wherein execution of the instructions causes the system to:
estimate an edge of one or more cartilage regions within an MRI segment associated with the selected joint;
determine a skeleton associated with the selected joint;
determine a volume based on the estimated edge and skeleton; and
determine the thickness associated with the joint based on the volume, summed over the MRI segment.

67. The non-transitory computer-readable storage medium of claim 55, wherein the three-dimensional diagnostic images include a bone edema and inflammation image.

68. The non-transitory computer-readable storage medium of claim 67, wherein the bone edema and inflammation image is based at least in part on a determination of a water concentration in one or more tissues associated with the selected joint.

69. The non-transitory computer-readable storage medium of claim 55, wherein the three-dimensional diagnostic images include a joint space width image.

70. The non-transitory computer-readable storage medium of claim 69, wherein execution of the instructions causes the system to determine a mean value from a lowest five percent distribution of joint spaces.

71. The non-transitory computer-readable storage medium of claim 55, wherein the three-dimensional diagnostic images include a bone spur identification image.

72. The non-transitory computer-readable storage medium of claim 55, wherein execution of the instructions causes the system to determine a water concentration of bones and cartilage associated with the selected joint based at least in part on a determination of a uniformity of voxel intensity.

73. The non-transitory computer-readable storage medium of claim 72, wherein the determination of the uniformity includes a determination of an entropy associated with one or more three-dimensional models.

74. The non-transitory computer-readable storage medium of claim 72, wherein the determination of the uniformity includes a determination of energy associated with voxels of one or more three-dimensional models.

75. The non-transitory computer-readable storage medium of claim 72, wherein the determination of the uniformity includes a determination of a gray level co-occurrence matrix of joint entropy.

76. The non-transitory computer-readable storage medium of claim 72, wherein the determination of the uniformity includes a determination of a gray level co-occurrence matrix of inverse difference.

77. The non-transitory computer-readable storage medium of claim 55, wherein execution of the instructions causes the system to:
determine quantitative joint information based at least in part on the three-dimensional models; and
display the quantitative joint information.

78. The non-transitory computer-readable storage medium of claim 55, wherein execution of the instructions causes the system to:
predict joint-related conditions based at least in part on the three-dimensional diagnostic images; and
display an image showing, at least in part, the predicted joint-related conditions.

79. The non-transitory computer-readable storage medium of claim 78, wherein the instructions to predict further include instructions to determine a classification of the predicted joint-related conditions.

80. The non-transitory computer-readable storage medium of claim 79, wherein the classifications include pain progression, joint space width progression, pain and joint space width progression, neither pain nor joint space width progression, or a combination thereof.

81. The non-transitory computer-readable storage medium of claim 55, wherein the instructions to predict are based on a deep-learning model executed by a trained convolutional neural network.
